(19) United States
(12) Patent Application Publication
(10) Pub. No.: US 2005/0255590 A1
(43) Pub. Date: Nov. 17, 2005

(54) Methods for generating neuronal cells from human embryonic stem cells and uses thereof

(76) Inventors: Ronald S. Goldstein, Jerusalem (IL); Benjamin E. Reubinoff, (US)

Correspondence Address: MORGAN & FINNEGAN, L.L.P., 3 World Financial Center, New York, NY, 10281-2101, US

(21) Appl. No.: 11/102,375

(22) Filed: Apr. 8, 2005

Related U.S. Application Data
(60) Provisional application No. 60/561,147, filed on Apr. 9, 2004.
(60) Provisional application No. 60/650,694, filed on Feb. 7, 2005.

(51) Int. Cl. C12N 5/08

(52) U.S. Cl. 435/368

(57) ABSTRACT

This invention relates generally to the production of human neuronal cells from human embryonic stem cells and/or human neuronal progenitor cells. In some embodiments, the human neuronal cells are neural crest cells. In other embodiments, the human neuronal cells are peripheral neurons. In other embodiments, the human neuronal cells are schwann cells. The invention provides methods of culturing and purifying human neuronal cells and uses thereof. Such uses include generating models of neuropathy, drug screening methods, and cell based therapeutic.

This application claims the benefit of priority under 35 U.S.C. § 119(e) of U.S. Provisional Application Nos. 60/561,147, filed Apr. 9, 2004, and 60/650,694, filed Feb. 7, 2005, the contents of each of which are hereby incorporated by reference in their entireties.

### FIELD OF THE INVENTION

This invention relates generally to production of human neural crest cells and human neuronal cells from human embryonic stem cells. Method of culturing and isolating the human neural crest cells and human neuronal cells in vitro are also encompassed. Methods of use of cells of the invention in cell-based treatments for neuropathy, including familial dysautonomia, are also encompassed. Models of neuropathy can be generated using human neural crest cells and human neuronal cells of the invention and used for, e.g., drug screening. The present invention also encompasses methods of production of neural cells from the differentiation of neuronal progenitor cells and/or neural crest cells of the invention.

### BACKGROUND ART OF THE INVENTION

Several central nervous system (CNS) cell types with potential clinical importance have been produced from embryonic stem cells, including neuronal progenitor cells, dopaminergic and cortical pyramidal-like neurons, spinal motoneurons, and oligodendrocytes. Despite these developments, however, understanding of the pathogenesis of and drug discovery/improvement for peripheral neuropathies will require the continuous production of peripheral neurons from human embryonic stem cells (HESC) or human neural progenitors (HNPr) in vitro.

A variety of studies using mouse embryonic stem cells have reported differentiation into neural progenitor cells, CNS neurons, and glia, in vitro. (Bain et al., “Neural differentiation of mouse embryonic stem cells,” Dev. Biol., 168(2):342-57, 1995; Strubing et al., “Differentiation of pluripotent embryonic stem cells into the neuronal lineage in vitro gives rise to mature inhibitory and excitatory neurons,” Mech. Dev., 53(2):275-87, 1995; Fraichard et al., “In vitro differentiation of embryonic stem cells into glial cells and functional neurons,” J. Cell Sci., 108(Pt10):3181-8, 1995; Okabe et al., “Development of neuronal precursor cells and functional postmitotic neurons from embryonic stem cells in vitro” Mech. Dev., 59(1):89-102, 1996; Lee et al., “Efficient generation of midbrain and hindbrain neurons from mouse embryonic stem cells,” Nat. Biotechnol., 18(6):675-9; Li et al., “Generation of purified neural precursors from embryonic stem cells by lineage selection,” Curr. Biol., 8(17):971-4, 1998; Kawasaki et al., “Induction of midbrain dopaminergic neurons from ES cells by stromal cell-derived inducing activity,” Neuron, 28(1):31-40, 2000; Wichterle et al., “Directed differentiation of embryonic stem cells into motor neurons,” Cell, 110(3):385-97, 2002).

There are also reports that HESC have been differentiated into neural progenitors and CNS neurons, in vitro. (Reubinoff et al., “Neural progenitors from human embryonic stem cells,” Nat. Biotechnol., 19:1129-33, 2001; Reubinoff et al., “Embryonic stem cell lines from human blastocysts: somatic differentiation in vitro,” Nat. Biotechnol., 18:399-404, 2000; Schuldiner et al., “Induced neuronal differentiation of human embryonic stem cells,” Brain Res., 913:201-05, 2001; Zhang et al., “In vitro differentiation of transplantable neural precursors from human embryonic stem cells,” Nat. Biotechnol., 9:1129-33, 2001; Rathjen et al., “Directed differentiation of pluripotent cells to neural lineages: homogeneous formation and differentiation of a neurectoderm population,” Development, 129: 2649-61, 2002).

In most of the protocols cited, differentiation of embryonic stem cells typically resulted in cell aggregates known as embryoid bodies (EB), within which a number of ectodermal, mesodermal, and endodermal derivatives were found. After EB formation, these were typically treated with the developmental morphogenic, retinoic acid, at superphysiological concentrations to promote neural differentiation. Plating of the EB on an adherent substrate promotes further differentiation and migration of post-mitotic neurons away from the EB. This protocol has generally been reported to yield a relatively low fraction of neurons, and most of these neurons had a GABAergic neurotransmitter phenotype (where GABA stands for γ-aminobutyric acid) (for a review see Stavridis and Smith, “Neural differentiation of mouse embryonic stem cells,” Biochem. Soc. Trans., (Pt. 1):45-49). The use of retinoic acid treatment of EB to obtain neurons is problematic for a number of reasons. It is difficult to control and analyze the steps involved in differentiation using this method, because EB contain multiple cell lineages. Furthermore, retinoic acid is a strong teratogen that can perturb neural patterning and neuronal identities in EB as it does in vivo (Soprano and Soprano, “Retinoids as teratogens,” Annu. Rev. Nutr., 15:111-32, 1995; Sucov and Evans, Retinoic acid and retinoic acid receptors in development. Mol Neurobiol., 10(2-3):169-84). Thus, the efficient generation of specific neuronal subtypes necessitated the development of alternative methods for neural differentiation of embryonic stem cells.

Important technical advances for the efficient generation of neuronal cells in large quantitities include the use of cell lines capable of inducing differentiation of murine and non-human primate embryonic stem cells without the formation of EB or the use of retinoic acid.

Although there has been considerable progress in the generation of CNS neural progenitor cells and neurons from mouse and human embryonic stem cells, the differentiation of embryonic stem cells into a wider range of neural lineages, including peripheral, post-mitotic neurons has only recently been reported in mouse embryonic stem cells and monkey embryonic stem cells: Mizuseki et al., Generation of neural crest-derived peripheral neurons and floor plate cells from mouse and primate embryonic stem cells. Proc. Natl. Acad. Sci. U. S. A., 100(10):5828-33, 2003.

Peripheral neuropathies refer to a syndrome of sensory loss, muscle weakness, muscle atrophy, decreased deep-tendon reflexes, and/or vasomotor symptoms. One type of peripheral neuropathy that is genetically inherited is familial dysautonomia (FD) (MIM#2239001), also known as Riley Day syndrome or hereditary sensory and autonomic neuropathy III (HSAN-III). FD is the best-known and most common member of a group of congenital sensory and autonomic neuropathies (HSN) characterized by widespread sensory and variable autonomic dysfunction (Axelrod F. B.: “Autonomic and Sensory Disorders,” In: Principles and Practice of Medical Genetics, 3^{rd} edition, A. E. H. Emory and D. L. Rimoin eds; Churchill Livingstone, Edinburgh. (1996) pp. 397-411).

FD is caused by a mutation in the IκB Associated Protein gene (IκBKAP), in which an intron 20 mutation (IVS20^{6T→C}) results in a unique pattern of tissue-specific exon 20 skipping. As a consequence of this mutation, the nervous system has a particularly low level of correctly spliced (exon-20-inclusive) IκBKAP transcript, and thus very low levels of functional IκBKAP protein are made. Insufficient levels of functional IκBKAP protein ultimately lead to apoptotic cell death.

### SUMMARY OF THE INVENTION

This invention relates to methods for generating human neural crest cells (HNCC), human peripheral neural cells (HPN), human Schwann cells (HSC), and/or other intermediate neuronal cell types by inducing differentiation of human embryonic stem cells (HESC). Neural differentiation of HESC results from contacting the HESC with a neural differentiation-inducing activity (NDIA) including, but not limited to stromal-derived inducing activity (SDIA). When done in vitro, the HESC and an entity which has NDIA are present under cell culture conditions that prevent formation of large cell aggregates. HESC/NDIA co-cultures may be cultured for a period of time such that HPN or HSC are differentiated. Alternatively, HESC/NDIA co-cultures may be cultured for an intermediate period of time such that HNCC are present and can be isolated prior to differentiation into HPN or HSC.

The present invention also relates to methods generating HNCC, HPN, HSC, and/or other intermediate neuronal cell types by inducing differentiation of human neural progenitors (HNPr) including, but not limited to neurospheres (NS).

The present invention also relates to methods of production of neural crest cell-derived cells from the differentiation of HNCC of the invention. Neural crest cell-derived cells can be differentiated from HNCC in vitro or in vivo. In specific embodiments, neural crest cell-derived cells include, but are not limited to, neurons, glia (e.g., schwann cells and satellite cells), secretory cells of the peripheral neuroendocrine system, melanocytes, chondrocytes, and/or smooth myocytes.

This invention also provides a method of purifying subpopulations of cells derived from the HESC or HNPr cells. In one embodiment, one or more monoclonal antibodies specific to the desired cell type are incubated with the cell population and those bound cells are isolated. In another embodiment, the desired subpopulation of cells express a reporter gene that is under the control of a cell type specific promoter. In a specific embodiment, the hygromycin B phosphotransferase-EGFP fusion protein is expressed in a cell type specific manner. The method of purifying comprises sorting the cells to select green fluorescent cells and reiterating the sorting as necessary, in order to obtain a population of cells enriched for cells expressing the construct (e.g., hygromycin B phosphotransferase-EGFP) in a cell-type-dependent manner.

This invention also provides for methods of treatment of disorders associated with deficient or defective HNCC, HPN, HSC and/or other intermediate neuronal cell types including, but not limited to peripheral neuropathies and disorders associated with CNS or PNS myelin degeneration. In one embodiment, HNCC, HPN, HSC and/or other intermediate neuronal cell types can be made and isolated using methods of the invention and introduced into an individual in need thereof. In another embodiment, HESC, HNPr, or HNC can be introduced into an individual in need thereof and differentiated into the desired cell type in vivo using the methods of the invention.

This invention also provides a method for generating an in vitro model of disorders associated with deficient or defective HNCC, HPN, HSC and/or other intermediate neuronal cell types including, but not limited to FD and disorders associated with CNS or PNS myelin degeneration. In one embodiment, HESC and/or HNPr can be genetically modified to comprise the mutation or mutations associated with a disorder. The mutation can be incorporated into the genome or be introduced by a minigene. In a specific embodiment, when the disorder is FD, the mutation is an IVS20^{+6T→C} transversion the IκBKAP gene. In another embodiment, expression of a gene associated with a disorder is altered (e.g., increased or decreased). Any method can be used to alter expression including, but not limited to, siRNA to decrease expression. In a specific embodiment, when the disorder is FD, expression of the IκBKAP gene is decreased. The modified cells are then differentiated into the desired cell type (i.e., HNCC, HPN, and/or HSC). The resulting differentiated cells comprise the mutation or altered expression levels and thus recapitulate the disorder.

This invention also relates to methods for generating an in vitro screen for agents that can alter the phenotype of a neuronal cell produced by the methods of the invention. In one embodiment, the neuronal cell used in the screen is a wild type cell. In another embodiment, the neuronal cell is an altered cell including, but not limited to, those mutant neuronal cells or neuronal cells with altered expression levels described supra. In a specific embodiment, the screen is used to identify agents that restore altered neuronal cell phenotype to a substantially wild type phenotype. In a preferred embodiment, the mutant phenotype is a IVS20^{+6T→C} transversion in the IκBKAP gene and the wild type phenotype is inclusion of exon 20 in the IκBKAP polypeptide in peripheral HPN.

As used herein, the term “neural” includes both neurons and glia.

As used herein, the term “peripheral neural cells” includes sensory neurons, sympathetic neurons, and glial cells (including ganlionic satellite cells, myelinating glia, and non-myelinating glia).

### BRIEF DESCRIPTION OF THE DRAWINGS

FIG. 1 Induction of ectodermal differentiation of HESC by PA6 cells. A colony of HESC induced by SDIA for 7 days was immunostained for (A) NCAM and (B) E-Cadherin. (C) In the merge of A and B shown in (C), the E-cadherin putative epithelial cells were seen to occupy the center of the colony, a pattern that was commonly found in the cultures. Panel (D) shows a colony from a 7-day co-culture double-stained for NCAM and AP2, a combination thought to be indicative of neural crest cells. By three weeks of culture, massive neuronal differentiation was observed in a majority of the colonies, as shown by the neuron-specific tubulin immunostaining shown in panels E and F. In panels E and F nuclei were stained blue with Hoechst. Bars=100 μm in A-E, and 50 μm in F.

FIG. 2 Induction of human peripheral neuron-like cells by PA6. A 4-week colony of SDIA-treated HESC stained for general neuronal marker β-III-tubulin and peripheral neuron marker peripherin is shown in (A). Arrowheads point to the processes of a bipolar cell double-stained for these markers. In other parts of the culture, large numbers of axons extending out of a colony were stained for peripherin as shown in (B). SDIA treatment induced large numbers of cells that express tyrosine hydroxylase (TH), as seen by the green staining in panel (C). (D-F) Some TH+ neurons were CNS-like, and some were HPN-like. A pair of TH+cells is shown in (D). Only one of these (arrow) also stained for peripherin (E), and was a putative sympathetic ganglion-like (SG) neuron. (F) shows a merge of (D) and (E). (G-I) Some peripherin+ cells were not TH+. Several TH+ cells are shown by arrows in (G). These cells were SG-like, but the same field contains some peripherin+ axons that are not TH+ (H). (I) shows a merge of G and H. Bars=100 μm in A-C, 30 μm in D-I.

FIG. 3 SDIA-treatment induces peripheral sensory-like neurons from HESC. A field in a co-culture of HESC with PA6 cells with a large number of Brn3a+ nuclei and peripherin+ axons is shown at low magnification in (A). Nuclear staining of the same field (B) shows both the PA6 feeder cells and large colony of HESC (asterisk). Note that the Brn3a+ nuclei in (A) are outside and at the edges of the colony. A portion of a merge of panels A and B is shown in (C). (D) shows the edge of another colony (asterisk) that had a small mass of peripherin+/Brn3a+neurons adjacent to it. In (E), a triplet of sensory-like neurons is indicated by the arrow. The arrow in (F) shows a cell with the morphology of a dorsal root ganglion “intermediate neuroblast”. β-3-tubulin stained cells with both bipolar (filled arrow) and pseudounipolar (open arrow) morphology are shown in (G). (H) is a collection of drawings by His of Golgi-stained developing dorsal root ganglion neurons for comparison to the stained cells in E-G. Bars=A&B 100 μm, C-H 50 μm.

FIG. 4 Quantitation of HESC colonies containing Brn3a and peripherin-immunoreactive cells after 4 weeks of SDIA induction. The percentage of colonies which contained no peripherin+ or Brn3a+ cells ( - - - ), singly-stained Brn3a+ cells or peripherin+ cells, or double-stained peripherin+/Brn3a+ cells is depicted. Results are the average of three experiments including a total of more than 300 colonies. Error bars=SEM.

FIG. 5 Temporal expression pattern of genes characteristic of peripheral sensory neurons in SDIA-induced HESC cultures. TrkC mRNA was expressed at a high levels at one week of SDIA treatment as compared to cells at three weeks of SDIA treatment. By contrast, in the chick embryo TrkA was only expressed in more mature DRG cells, and its mRNA increased over time from 1 to 3 weeks of SDIA treatment. Transcripts for the intermediate filament protein characteristic of peripheral neurons, peripherin, were only detected from 3 weeks of culture, consistent with our immunocytochemical evidence for the appearance of the protein at about this time.

FIG. 6 Temporal expression pattern of genes characteristic of neural crest in SDIA-induced HESC cultures. Several genes that were used as markers of the neural crest phenotype in non-primate species (see text) were observed to have a pattern of expression consistent with the generation of a neural-crest cells in SDIA-induced HESC cultures. The genes Snail, Sox9, Msx1, and dHAND were all increased at 1 week as compared to naïve HESC. At three weeks, the genes Snail, Sox9, Msx1, and dHAND were down-regulated as cells further differentiated. Another gene used as a marker for neural crest cells, FoxD3 was expressed by HESC at similar levels in naïve HESC and at 1 and 3 weeks of PA6 co-culture. AP2 expression increased from 1 week to 3 weeks of co-culture, consistent with its expression in both epidermal precursors and neural crest cells. The expression pattern of AP2 was the same as was observed for the epithelial marker E-cadherin.

FIG. 7 Temporal expression pattern of genes characteristic of schwann cells in SDIA-induced HESC cultures. Protein zero mRNA, a schwann cell specific transcript, was present at one week of SDIA treatment and increased at three weeks SDIA treatment.

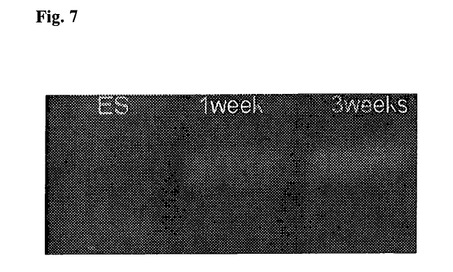

FIG. 8 pN-Select, a hygromycin B phosphotransferase-EGFP fusion protein expression vector in which a cell-type-specific promoter is used to drive expression of the bifunctional selection marker-fluorescent reporter protein only in HESC undergoing neural differentiation. The vector is useful for selecting particular cell types with selection by hygromycin and/or FACS sorting of cells based on EGFP fluorescence.

### DETAILED DESCRIPTION OF THE INVENTION

This invention provides novel methods for efficient generation of human neural crest cells (HNCC), human peripheral neurons (HPN), human schwann cells (HSC) and other intermediate cell types derived from the differentiation of human embryonic stem cells (HESC) and/or human neural progenitor cells (HNPr) such as neurospheres. The HESC and/or HNPr for use in the methods of the invention can be a primary cells or a cell line. In preferred embodiments, the generation of NC, HPN, HSC, and/or other intermediate cell types are performed in vitro. The methods of the invention can be used to generate substantially purified populations of NC, HPN, HSC, and/or other intermediate cell types. Alternatively, the methods of the invention encompass the formation of a mixture of NC, HPN, HSC, and/or other intermediate cell types that is derived from the differentiation of HESC and/or HNPr. Methods of isolating substantially purified population of NC, HPN, HSC, and/or intermediate cell types thereof are also encompassed.

In preferred embodiments, HESC and/or HNPr are differentiated by contact with a neural differentiation inducing activity (NDIA). In more preferred embodiments, the NDIA is a stromal-derived inducing activity (SDIA). In such embodiments, HESC and/or HNPr are co-cultured according to the methods of the invention with a stromal cell line or derivative thereof effective for inducing neural differentiation of NC, HPN, HSC, and/or other desired intermediate cell types. In a more preferred embodiment, the SDIA is the PA6 stromal cell line or a derivative thereof. Derivatives of stromal cell lines include, but are not limited to, a membrane preparation of a cell line possessing SDIA, non-viable stromal cell line possessing SDIA wherein the cell line has been histologically fixed, irradiated, or inhibited from going through mitosis.

The invention also relates to methods of production of neural crest cell-derived cells from the neural crest cell of the invention. Neural crest cell-derived cells can be differentiated from HNCC in vitro or in vivo. In specific embodiments, neural crest cell-derived cells include, but are not limited to, neurons, glia (e.g., schwann cells and satellite cells), secretory cells of the peripheral neuroendocrine system, melanocytes, chondrocytes, and/or smooth myocytes.

This invention also provides for methods of treatment of disorders associated with deficient or defective HNCC, HPN, HSC and/or other intermediate neuronal cell types including, but not limited to peripheral neuropathies and disorders associated with CNS or PNS myelin degeneration.

This invention also provides a method for generating an in vitro model of disorders associated with deficient or defective HNCC, HPN, HSC and/or other intermediate neuronal cell types including, but not limited to familial dysautonomia (FD) and disorders associated with CNS or PNS myelin degeneration. The models of the disorder can be used to screen for agents that can alter cell phenotype.

Methods for Generation of Neural Crest Cells, Peripheral Neurons, and Schwann Cells

NC, HPN, HSC, and/or other intermediate cell types thereof can be prepared according to methods of this invention by contacting undifferentiated HESC and/or HNPr with an NDIA sufficient to differentiate HESC and/or HNPr into cells of neural lineage. NDIA may be provided by many different methods. In preferred embodiments, the NDIA is a stromal-derived inducing activity (SDIA). In such embodiments, HESC are co-cultured according to the methods of the invention with a stromal cell line or derivative thereof effective for inducing neural differentiation of NC, HPN, and/or other desired intermediate cell types. Any stromal cell line known in the art that exhibits SDIA can be used (see, e.g., those disclosed in Kawasaki et al., 2000, Neuron 28:31-40). In a more preferred embodiment, the SDIA is the PA6 stromal cell line or a derivative thereof.

Derivatives of stromal cell lines include, but are not limited to, a membrane preparation of a cell line possessing SDIA and non-viable whole cells possessing SDIA wherein the cell line has been histologically fixed (such as with paraformaldehyde) or mitotically arrested (such as by treatment with mitomycin C or irradiation by γ-irradiation).

In one embodiment, HESC colonies are first separated from a fibroblast feeder layer. Separation may be facilitated by use of a proteolytic enzyme such as trypsin, for example, to gently separate the HESC colonies. The HESC colonies are then disaggregated into a cell suspension by adequate titration. HESC in the cell suspension are counted subsequently and seeded on an entity possessing NDIA (including, but not limited to stromal cell lines such as PA6) at a density of approximately 1000 cells/cm². In embodiments a where the NDIA is supplied by stromal cells, optionally, prior to co-culture with HESC, the stromal cells are mitotically arrested.

Co-culture of HESC and cells possessing NDIA is initiated in a growth medium, preferably including the following components: BHK-21 medium/Glasgow MEM or similar cell culture medium, and preferably 10% Knockout serum replacement, 2 mM glutamine, 1 mM pyruvate, 0.1 mM non-essential amino acids, and 0.1 mM β-Mercaptoethanol. Based on the cell culture conditions described herein, one skilled in the art can make modifications in order to adapt to various cell growth conditions and requirements. Medium is replenished sufficiently to maintain cell viability, preferably by replacing the medium at appropriate intervals (e.g., on days 1, 4, and 6). On or about day 8 of co-culture of HESC with cells possessing NDIA, the medium is replaced with a serum-free medium including, but not limited to the following: BHK-21 medium/Glasgow MEM, 100 μM tetrahydrobiopterin, 1 mM pyruvate, 0.1 mM non-essential amino acids, 2 mM glutamine, 0.1 mM β-Mercaptoethanol, Tryptose Phosphate, and N2 supplement (Gibco).

In one specific embodiment, co-culture in the described serum-free medium is continued for a time sufficient for maximum differentiation of HESC into HPN and/or HSC (preferably at least 20 days).

In another specific embodiment, co-culture in the described serum-free medium is continued for a time sufficient for maximum differentiation of HESC into HNCC (preferably 6-8 days).

In another specific embodiment, intermediate periods of co-culture may be used to obtain cells having a neural phenotype intermediate (between HESC and HPN/HSC or between HNCC and HPN/HSC).

Further, differentiation of HNPr by the methods described herein gives rise to post-mitotic peripheral neurons of various lineages. These peripheral neurons of various lineages optionally can be characterized by cell-type-specific expression of marker proteins as described herein. Production of HPN by differentiation of HNPr has the advantage of being more rapid than the procedure for obtaining peripheral neurons starting from HESC. In some embodiments HNCC are used as the HNPr used to generate HPN.

In another embodiment, HNPr are co-cultured with the entity that posses NDIA. HNPr are self-renewing and multipotent, having the ability to give rise to several different neural lineages when subject to differentiation methods as described herein. A variety of HNPr sources can be used, including differentiation of HESC (for example by the methods described herein), human embryonic tissue, or adult human tissue. Such cells are typified by expression of marker proteins such as Sox 1. Production of HPN, HSC and/or intermediate cell types by differentiation of HNPr has the advantage of being more rapid than the procedure starting from HESC and may reduce non-neuronal cell yield.

In a specific embodiment, the HNPr are neurospheres. Neurospheres are balls of neural precursors that grow in suspension culture and are passaged by mechanical cutting or breaking with a pipette. They were originally made from embryonic neural tube but have since been derived from many sources, including adult human spinal cord and brain. The neurospheres are grown in a solution of growth factors and mitogens (e.g., noggin) which allows their expansion (see e.g., Rao, 2004, J. Neurotrauma. 21:415-27; U.S. Pat. No. 6,875,607, U.S. patent Publication 2002/0164308). To differentiate neurospheres, the cells are trypsinisated to a single cell suspension and plated on lamin/fibronectin/polylysine substrates or an entity that posses NDIA (e.g., PA6 cells). Cells are incubated in serum free medium containing NGF and B27 supplement. Medium is replaced every 3 days. After 7 days of co-culture with PA6 cells, a morphological change could be observed. After 26 days of co-culturing neural differentiation is seen.

In another specific embodiment, the HNPr are HNCC. HNCC are co-cultured with an entity that posses NDIA to yield HPN, HSC, and/or other intermediate cell types thereof. Time periods for co-culture incubation can be adjusted accordingly from those described supra for HESC co-culture.

In some embodiments, one or more additional factors can be added to the co-culture of HESC and/or HNPr to alter the speed of differentiation and/or the type of differentiation (e.g., what types of cells result). The factors may be added at any time during co-culture. In one embodiment, bone morphogenic protein 4 (BMP4) is added to the co-culture (e.g., about a week after co-culture). BMP4 is used at low concentrations (about 0.5 nM) for the culture of sensory neurons and high concentrations (about 5 nM) for sympathetic neurons. In another embodiment, other factors that can be added to the co-culture nearer to the end of differentiation to increase the viability of the differentiated cells. Such factors include, but are not limited to, Nerve Growth Factor (NGF), Brain-Derived Neurotrophic Factor (BDNF), Neurotrophin 3 (NT3), Ciliary Neurotrophic Factor, Glial Cell-Derived Neurotrophic Factor (GDNF), and Wnt-1. In another embodiment, the secreted protein Noggin (Valenzuela et al, 1995, J Neurosci. 15:6077-84) is added to cell culture medium shortly after the beginning of the co-culture. In another embodiment, factors can be added to increase the yield of peripheral ganglion neurons including, but not limited to, BMP4, wnt, and retinoic acid.

In other embodiments, the expression of one or more cell-expressed factors are altered in order to alter the speed of differentiation and/or the type of differentiation (e.g., what types of cells result). In such embodiments, the HESC or HNPr have been modified prior to co-culture such that a cell-expressed factor displays an altered expression level, expression pattern, and/or time period of expression. In a specific embodiment, factors can overexpressed include, but not limited to, NCX, snail, FoxD3, Sox 9, beta-catenin, and neuregulin (GCF) (see, e.g., Bronner-Fraser et al., 2004, Science 303:966-968 and Meulemans, 2004, Developmental Cell 7: 291-299).

Differentiation of HESC and/or HNPr results in HNCC, HPN, HSC, and/or other intermediate cell type which are characterized by expression of one or more cell-type specific markers or a profile of markers that are sell-type specific. Cell-type specific markers or profiles include, but are not limited to, peripherin/Brn3a for sensory neurons; peripherin/dopamine beta hydroxylase or peripherin/tyrosine hydroxylase for sympathetic neurons; Snail, Sox 9, Msx 1, dHAND, and low affinity NGF receptor (p75) for HNCC; protein zero for schwann cells. Expression of these proteins can be detected by a number of methods known to the art including, for example, immunofluorescence, ELISA, or RT-PCR.

Methods for Purifying HNCC, HPN, and HSC

The present invention encompasses populations of HNCC, HPN, HSC, and/or other intermediate cell type which are substantially purified and methods of purifying the same. HNCC, HPN, HSC, and/or other intermediate cell types can be purified from a population of cells comprising the desired cell type that has been derived from the differentiation of HESC or HNPr by any method known in the art. In one embodiment, the desired cell type is isolated by contacting a population of cells comprising the desired cell type with one or more monoclonal antibodies, each of which binds to a cell type-specific factor for the desired cell type (preferably on the cell membrane), under conditions sufficient for binding. In embodiments where the cells to be isolated are HNCC, cell type specific markers include, but are not limited to, low affinity NGF receptor (p75), Snail, Sox 9, Msx 1, NCX, and/or dHAND. In embodiments where the cells to be isolated are HSC, cell type specific markers include protein zero. Cells bound to the one or more antibodies are isolated by any method known in the art. For example, the cells can be further incubated with a second antibody that is fluorescent conjugated and binds to the antibody bound to the cell type-specific factor and subjected to FACS analysis. Alternatively, the cells are further incubated with an antibody that binds to the antibody bound to the cell type-specific factor, wherein the second antibody is attached to a solid matrix (e.g., magnetic beads or matrix of a column). Cells bound to the solid matrix can be isolated.

In a specific embodiment, the cell type-specific maker is a profile of markers that is specific for the desired cell type. Methods disclosed above can be modified such that the sub population of cells expressing the profile of markers are preferentially isolated.

In another embodiment, HNCC, HPN and/or HSC are isolated by preferential expression of a marker gene under the control of a cell type-specific promoter. HESC and/or HNPr are stably transfected with a selection marker-reporter expression cassette that is under the control of a cell-type-specific promoter. These cells may be used as the starting cells for differentiation into HNCC, HPN and/or HSC by contact with a NDIA. Any gene which upon expression provides a mechanism for selecting for transfected cells is suitable as a selection marker gene. The reporter component of the expression cassette comprises a gene which upon expression provides a means for detecting the presence of the transferred gene. As stated above, expression of cell type specific proteins may also be used to select for cells expressing the desired phenotype. In preferred embodiments, HNCC, HESC and/or HNPr stably transfected with a selection marker-reporter expression cassette under the control of a cell type-specific promoter are exposed to a selection agent several days after a reporter activity (e.g. fluorescence) is first detected. Optionally, when live stromal cells are used as NDIA in the methods of differentiation, the stromal cells may be stably transfected with selection marker genes which may be constitutively expressed and confer resistance to selection agents used during the differentiation procedure described herein.

In a specific embodiment, HESC and/or HNPr that have been stably transfected with a cell-type-specific Hyg-EGFP expression cassette are first subjected to differentiation by the methods described herein. Differentiated HESC and/or HNPr are then exposed to hygromycin at a concentration effective for killing cells that do not express the Hyg-EGFP protein. As mentioned herein, expression of hygromycin B phosphotransferase-EGFP can be driven by different cell type-specific promoters and accordingly, different subpopulations of the differentiated cells will survive hygromycin treatment in each case. Treatment with hygromycin is continued until the majority of viable cells remaining are EGFP-positive. After selection in hygromycin, EGFP-positive cells are removed from the cell culture substrate and purified by FACS so as to generate a population of cells selected for a cell-type-specific expression phenotype.

In another specific embodiment, treatment with a selection agent such as hygromycin is omitted and differentiated cells are selected solely on the basis of fluorescence by FACS. In yet another embodiment, cells expressing a selectable-reporter gene are exposed to the appropriate selection agent without subsequently being purified by FACS.

As is well known in the art, the promoters of cell type-specific genes can be used to direct the expression of reporter genes and/or selection marker genes. Reporter genes include genes encoding a variety of proteins well known in the art, non-limiting examples of which include EGFP, enhanced yellow fluorescent protein, cyan fluorescent protein, red fluorescent protein, β-lactamase, and luciferase. It is understood that in cases where reporter protein activity requires addition of a substrate, such a substrate will be provided at an adequate concentration for detecting reporter activity. Selection marker genes are genes that enable survival of a population of cells that express the selection marker gene, when the cells are in the presence of the respective selection agent that is cytotoxic otherwise. Examples of selection agents and their respective selection marker genes, include, but are not limited to, neomycin and neomycin phosphotransferase; hygromycin and hygromycin B phosphotransferase; and puromycin and puromycin N-acetyl transferase.

Fusion proteins that are bifunctional with respect to selection agent resistance and a detectable (e.g., fluorescent) reporter activity can be generated using standard genetic engineering techniques. Examples of such bifunctional fusion proteins include, but are not limited to the following: hygromycin B phosphotransferase-EGFP, neomycin phosphotransferase-EGFP, puromycin N-acetyltransferase-EGFP, etc. These proteins are comprised of C-terminal fusions of the EGFP open reading frame to the open reading frame of the respective selection marker gene. In another embodiment of this invention, EGFP and a selection marker protein may be translated from separate open reading frames of a bicistronic mRNA, by linking the open reading frames together with an internal ribosomal entry site (IRES) sequence. In other embodiments of this invention, the selectable marker and reporter genes may be on separate constructs and not present as fusion proteins.

In some specific embodiments of the present invention, HESC and/or HNPr are generated which have one or more genomically integrated DNA constructs that encode a selectable marker-reporter protein that comprises (i) a cell-type-specific promoter operably linked to control expression of a bifunctional selection marker-reporter fusion gene wherein the cell-type-specific promoter remains inactive in undifferentiated HESC and/or HNPr and (ii) a constitutively active promoter that controls expression of a second selection marker gene, independently 6f cell-type. HESC and/or HNPr which have integrated the construct are selected by exposing cells to an appropriate selection agent, that is, one to which resistance is conferred by constitutive expression of the appropriate selection marker gene. For example, in one embodiment, the bifunctional selection marker-reporter gene is hygromycin B phosphotransferase-EGFP (Hyg-EGFP), the constitutively expressed selection marker gene is puromycin N-acetyltransferase, and the selection agent used for selection of stably transfected HESC is puromycin. In preferred embodiments, the DNA construct comprising a constitutively active promoter and a selection marker is separate (in trans) from the construct comprising a cell-type-specific promoter that controls expression of a bifunctional selection marker-reporter gene, as described herein. Non-limiting examples of the cell type-specific promoter that controls expression of the selection marker-reporter gene include the following: Sox 1 promoter, Sox 9 promoter, Neurogenin 1 promoter, Neurogenin 2 promoter, Peripherin promoter, Brn3a promoter, Snail, low affinity NGF receptor (p75), Msx 1, dHAND, and/or protein zero. DNA constructs described herein can be introduced into HESC and/or HNPr by a number of methods well known in the art, including electroporation, lipofection, and retroviral infection, including infection by lentiviruses (see, e.g., Gropp et al., “Stable genetic modification of human embryonic stem cells by lentiviral vectors,” Mol. Ther. February 2003; 7(2): 281-7). In a preferred embodiment, a modification of mouse ES cell electroporation is used that is suitable for stable transfection of HESC and/or HNPr, according to the method of Zwaka et al., (Nat. Biotechnol., 21:319-321, 2003). The modification includes (i) electroporating clumps of HESC and/or HNPr rather than single cell suspensions and (ii) electroporating the cells in an isotonic, protein-rich solution (e.g. serum-containing cell culture medium).

The purity of the population can assayed by determining the per cent of purified cells that express the cell type-specific marker or profile of markers.

Use of Neural Crest Cells for Further Differentiation

The present invention also relates to methods of production of neural crest cell-derived cells from the differentiation of HNCC of the invention. In one specific embodiment, neural crest cell-derived cells can be differentiated from HNCC in vitro. Any method known in the art for differentiating neural crest cell-derived cells can be used. Additional factors may or may not be added to the HNCC cells during differentiation. HNCC cells may or may not be modified to have altered expression of a cell-expressed factor.

In another specific embodiment, neural crest cell-derived cells can be differentiated from HNCC in vivo. The fate of HNCC is determined at least in part by their local environment (LeDouarin, 1980, Nature 286:663-9). Because of this, the area of the body where the HNCC are implanted can help determine what neural crest cell-derived cells the HNCC differentiate into. Additional factors may or may not be added to the HNCC cells during differentiation. HNCC cells may or may not be modified to have altered expression of a cell-expressed factor.

In specific embodiments, neural crest cell-derived cells include, but are not limited to, neurons, glia, secretory cells of the peripheral neuroendicrine system, melanocytes, chondrocytes, and/or smooth myocytes (see, e.g., LeDouarin, 1982, The Neural Crest, Cambridge, England:Cambridge University Press).

HNCC cells or cells differentiated from HNCC of the invention may be transplanted into a patient in need thereof for cell-based therapies. Any pathology related to deficient or defective HNCC or deficient or defective neural crest cell-derived cells can be treated by the implantation of HNCC or HNCC that have wholly or partially differentiated. HNCC cells or cells differentiated from HNCC of the invention may be used in screening assays for drugs that affect the etiology of pathology that results from deficient or defective HNCC or deficient or defective neural crest cell-derived cells.

Methods of Expanding Differentiated Neuronal Cells

Expansion of cells during the differentiation process prior to cell differentiation into the desired cell type can be used to increase the cell yield. In one embodiment, HNPr (e.g., HNCC) are isolated from differentiating HESC co-cultures and incubated with mitogens to increase cell number without substantially altering the state of differentiation. The expanded cells can then be returned to the differentiation conditions until the desired cell type has formed (i.e., HPN and/or HSC). In another embodiment, where HNCC are the desired cell type, the cells can be expanded as described supra and used according to the methods of the invention without returning to the differentiation conditions. Any mitogen known to effect the cell type to be expanded can be used. For example, EGF and FGF are HNCC mitogens and neuregulin/heregulin are schwann cell mitogens.

Transplantation of Purified Populations of Differentiated Neuronal Cells

This invention encompasses methods of treatment of disorders associated with deficient or defective HNCC, HPN, HSC and/or other intermediate neuronal cell types including, but not limited to peripheral neuropathies and disorders associated with CNS or PNS myelin degeneration. In one embodiment, HNCC, HPN, HSC and/or other intermediate neuronal cell types can be made and isolated using methods of the invention and introduced into an individual in need thereof. In another embodiment, HESC, HNPr, or HNC can be introduced into an individual in need thereof and differentiated into the desired cell type in vivo using the methods of the invention.

The transplantation of HPN or HNPr is a therapy for treatment of a peripheral neuropathy and/or neuronal injury, such as resulting from trauma. Examples of peripheral neuropathies that may be treated by the methods disclosed herein include, without limitation, peripheral neuropathies associated with acute or chronic inflammatory polyneuropathy, amyotrophic lateral sclerosis (ALS), Wallerian degeneration, distal axonopathy, collagen vascular disorder (e.g., polyarteritis nodosa, rheumatoid arthritis, or systemic lupus erythematosus), diphtheria, hereditary peripheral neuropathy (e.g., Charcot-Marie-Tooth disease (including type I, type II, and all subtypes), hereditary motor and sensory neuropathy (types I, II, and III, and peroneal muscular atrophy), hereditary neuropathy with liability to pressure palsy, infectious disease (e.g., AIDS), Lyme disease (e.g., infection with Borrelia burgdorferi), invasion of a microorganism (e.g., leprosy) leukodystrophy, metabolic disease or disorder (e.g., amyloidosis, diabetes mellitus, hypothyroidism, porphyria, sarcoidosis, or uremia), neurofibromatosis, nutritional deficiencies, paraneoplastic disease, peroneal nerve palsy, polio, porphyria, postpolio syndrome, Proteus syndrome, pressure paralysis (e.g., carpal tunnel syndrome), progressive bulbar palsy, radial nerve palsy, spinal muscular atrophy (SMA), a toxic agent (e.g., barbital, carbon monoxide, chlorobutanol, dapsone, emetine, heavy metals, hexobarbital, lead, nitrofurantoin, orthodinitrophenal, phenytoin, pyridoxine, sulfonamides, triorthocresyl phosphate, the vinca alkaloids, many solvents, other industrial poisons, and certain AIDS drugs), trauma (including neural trauma), and ulnar nerve palsy (Beers and Berkow, eds., The Merck Manual of Diagnosis and Therapy, 17^{th} ed. (Whitehouse Station, N.J.: Merck Research Laboratories, 1999) chap. 183). In a preferred embodiment of the present invention, the neuropathy is FD.

The transplantation of HSC or HNPr is a therapy for treatment of disorders associated with CNS or PNS myelin degeneration. Examples of the disorders include, but are limited to, multiple sclerosis, chronic inflammatory demyelnating polyneuopathy, and Guillain-Barre syndrome. Additionally, many myelin degeneration disorders of the CNS are autoimmune disorders. Peripheral nervous system myelin forming cells may not be recognized or may be recognized less well by the autoimmune machinery than CNS myelin forming cells.

Generation of an In Vitro Model of Neuropathies

This invention also provides in vitro models of peripheral neuropathies. In some embodiments, a human gene of interest can be specifically mutated within the genome of HESC and/or HNPr by use of “Knock-In” technologies originally developed in the art for manipulation of the genome of mouse embryonic stem cells. By changing the genotype of the HESC and/or HNPr through recombinant techniques, one or more mutations can be introduced which will result in phenotype changes associated with peripheral neuropathies. HESC and/or HNPr may be mutated so as to express mutations known to be associated with specific neurological disorders. HNCC, HPN, HSC, and/or intermediate cell types derived from these cells may then be used as model systems to investigate the phenotype of the cells and possible interventions to restore normal function. Alternatively, other mutations may be introduced which have not previously been identified with a particular phenotype as a means of investigating the role of specific genes in neuronal cell development and function.

In one embodiment, HESC and/or HNPr may be modified to possess a mutated IκBKAP gene which has been associated with familial dysautonomia (FD) (Slaugenhaupt et al., U.S. patent application 20020169299 which is incorporated by reference). In particular, the mutation in the endogenous IκBKAP gene is the IVS20^{30 6T→C} transversion mutation. Generation of a human embryonic stem cell knock-in cell line includes, in this embodiment, the steps of (i) generating a targeting vector comprising (a) a large genomic fragment of the IκBKAP gene in which the IVS20^{+6T→C} transversion mutation has been introduced by genetic engineering techniques, (b) a positive selection expression cassette located within the 5′ untranslated region of the gene (i.e., within a region of homology to the IκBKAP gene), wherein the positive selection expression cassette includes a constitutively active promoter which drives expression of neomycin phosphotransferase, and a negative-selection expression cassette located within the targeting vector backbone, but outside of the region of homology to the IκBKAP gene, wherein the negative selection expression cassette comprises a constitutively active promoter driving expression of herpes thymidine kinase; (ii) positive-negative selection wherein cells are first contacted with neomycin to (positively) select for cells that have genomically integrated the targeting vector described above, and subsequently cells are contacted with ganciclovir to select only cells in which the mutated IκBKAP gene sequence has become genomically integrated by homologous recombination thereby excluding the negative selection expression cassette. This procedure may be generalized to other mutated genes as well. In a preferred embodiment, increasingly higher concentrations of neomycin are used to contact transfected HESC and/or HNPr to favor selection of cells in which targeting of both alleles by homologous recombination has occurred. In an alternative embodiment, a negative selection cassette is omitted from the targeting vector and homologous recombination of the targeting vector in HESC and/or HNPr is detected by screening colonies of cells that survive positive selection. Screening of HESC and/or HNPr colonies includes isolating genomic DNA from the colonies, subjecting the genomic DNA to appropriate restriction digests, and detecting homologous recombination at the IκBKAP genomic locus by Southern blot analysis of genomic restriction digests or by a genomic PCR reaction that will detect integration of the targeting vector at the homologous locus.

HESC and/or HNPr knock-in cell lines, generated by the methods described herein, can be used as starting cell lines for the generation of HESC and/or HNPr stably transfected with a selection marker-reporter expression cassette under the control of a cell-type-specific promoter. The resulting embryonic stem cell lines will have a mutated IκBKAP gene and have a stably integrated selection marker-reporter gene. In a specific embodiment a purified population of HPN, homozygous for the IVS20^{+6T→C} transversion mutation and expressing a selection marker-reporter gene under the control of a cell-type specific promoter, can be obtained using the methods described in the present invention.

Alternatively, HESC and/or HNPr can be modified to alter expression levels of one or more polypeptides associated with a neuropathy. Any method known in the art can be used to alter expression. In one embodiment, a transgene under the control of a constitutive promoter is used to increase expression. In another embodiment, siRNA or antisense technology is used to decrease expression. Modified HESC and/or HNPr can be used in methods of the invention to differentiate into neuronal cells (e.g., HNCC, HPN, HSC, and/or intermediate cell types) that display the altered expression levels. In a specific embodiment, siRNA is used to decrease expression of the IκBKAP gene in HPN in models of FD.

Screening for Substances that Decrease Apoptosis of Human Peripheral Neurons

/As discussed above, in certain embodiments of this invention, HPN derived from HESC that express mutations known to be associated with a neurological disorder can be used as model systems. For example, the HPN can be used in a screening assay to screen for substances that decrease apoptosis of HPN. In a specific embodiment, the neurological disorder is FD, and the purified HPN that are homozygous for the IVS20^{+6T→C} transversion mutation in the IκBKAP gene (HPN^{+6T→C}) can be used to detect substances that decrease apoptosis of these HPN. In a preferred embodiment, purified HPN that are homozygous for the IVS20^{+6T→C} transversion mutation in the IκBKAP gene are plated in multi-well dishes appropriate for high-throughput screening and the HPN are contacted with test substances over a range of concentrations covering three orders of magnitude. The cells are contacted with test substances over a period of time within which neuronal apoptosis would normally occur in vitro for HPN carrying the FD IVS20^{+6T→C} mutation. A control group includes (HPN^{+6T→C}) that are contacted with a control substance not known to affect apoptosis of HPN (e.g. dimethyl sulfoxide). Apoptosis of (HPN^{+6T→C}) is measured in parallel for test substance and control substance groups. Apoptosis can be measured by various methods, including staining with fluorescent nuclear dye (Hoechst, propidium iodide), TUNEL staining, etc.

Screening for Substances that Increase the Ratio of Endogenous IκBKAP Gene IVS₂₀^{+6T→C} mRNA Including Exon 20 to IκBKAP Gene IVS20^{6T→C} mRNA Excluding Exon 20

Another embodiment of the invention uses HPN^{+6T→C} to detect substances that increase the ratio of IκBKAP gene IVS₂₀^{+6T→C} mRNA including exon 20 to IκBKAP gene IVS20^{+6T→C} mRNA excluding exon 20. Assays of mRNA levels are well known in the art and include, for example, quantitative reverse-transcription PCR assays, RNA blot assays, or RNAse protection assays. A specific embodiment includes (i) isolation of total RNA from HPN^{+6T→C} treated with a test substance or a control substance (ii) RT-PCR with primers that hybridize to target sequences which flank exon 20 of the IκBKAP gene, so that a PCR product of distinctly greater molecular size than the expected product size is generated by an mRNA template that includes exon 20, as compared to an mRNA template that excludes exon 20 (iii) quantification of the respective products using methods such as video quantification of electrophoretically separated PCR products.

Screening for Substances that Increase the Ratio of a IκBKAP Gene IVS20^{+6T→C} Minigene mRNA Including Exon 20 to IκKAP Gene IVS20^{+6T→C} mRNA Excluding Exon 20

In further embodiments of the invention, HESC which are wild type with respect to the endogenous IκBKAP gene, are used in transfection experiments. The experiments comprise transfection of a DNA construct that includes a vector backbone, a selection-agent resistance gene expression cassette, and an IVS20^{+6T→C} mutated IκBKAP minigene, which comprises exon 20 and its splice junctions. Transfection of the minigene may be transient, but for a period sufficient to quantitatively measure minigene mRNA transcript levels at any point during an experiment. In preferred embodiments, the transfection with the minigene is a stable transfection whereby a stably transfected human embryonic stem cell line is established (herein termed a minigene human embryonic stem cell line). Cells from a minigene human embryonic stem cell line can be used as the starting point for obtaining purified HPN. The majority of minigene mRNA transcripts in the HPN will exclude exon 20 due to missplicing caused by the IVS20^{+6T→C} mutation. HPN derived from a minigene human embryonic stem cell line can therefore be used to screen substances that can correct missplicing of the minigene mRNA transcripts, such that the ratio of the level of minigene mRNA transcript including exon 20 to the level of minigene mRNA transcript excluding exon 20 increases. Methods for measuring the ratio of the level of minigene mRNA transcript including exon 20 to the level of minigene mRNA transcript excluding exon 20 use steps similar to those described above, except that primers are designed to hybridize to target sequences within the vector backbone and not to hybridize with endogenous human embryonic stem cell sequences. In preferred embodiments, measurements of the ratio of the level of minigene mRNA transcript including exon 20 to the level of minigene mRNA transcript excluding exon 20 is made from cells exposed to a control substance that does not affect minigene mRNA transcript splicing. This method is advantageous, in that the minigene serves as a reporter of missplicing without causing other cellular phenotypes associated with IκBKAP gene missplicing.

The contents of all published articles, books, reference manuals and abstracts cited herein, are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Modifications and variations of the present invention are possible in light of the above teachings.

### EXAMPLES

### Example 1

### Generation of Peripheral Sensory Neurons, Sympathetic Neurons, and Neural Crest Cells from Human Embryonic Stem Cells

Materials and methods

Cell culture

Human embryonic stem cells [HES-1 (XX) (12) and HUES 7 XY and HUES 1 (XX) (13) cell lines] were cultured on mitotically-inactivated mouse embryonic or human neonatal fibroblast feeder layers in gelatin-coated tissue culture dishes and passaged every 6-7 days in 80% knock-out DMEM supplemented with, 20% knock-out serum replacement, 1 mM glutamine, 1% non-essential amino acids, units/ml penicillin, 50 μg/ml streptomycin, 0.1 mM β-mercaptoethanol, and 4 ng/ml b-FGF. The mouse PA6 cell line, obtained from the Riken Cell Bank (Riken, Japan), was cultured on gelatin-coated dishes in 90% DMEM, 10% fetal calf serum, 4.5 gm/l D-glucose, 1 mM L-glutamine, 75 units/ml penicillin, and 75 μg/ml streptomycin.

SDIA Induction

Approximately 10⁴ PA6 cells were seeded on gelatin-coated 13-mm coverslips in 24 well dishes. Confluent cultures, were treated with trypsin/EDTA for 4 min and the fibroblast feeder layer was removed manually, leaving the HESC colonies. The colonies were then triturated, and approximately 1000 HESC cells were placed in each PA6-containing well. The medium was then changed to 90% BHK-21 medium/Glasgow MEM, 10% Knockout serum replacement, 2 mM glutamine, 1 mM pyruvate, 0.1 mM non-essential amino acid solution, and 0.1 mM β-mercaptoethanol. Medium was changed 4 and 6 days after HESC plating. On day 8, the medium was changed to 90% BHK-21 medium/Glasgow MEM, 100 μM tetrahydrobiopterin, 2 mM glutamine, 1 mM pyruvate, 0.1 mM non-essential amino acid solution, N2 supplement X1, and 0.1 mM β-mercaptoethanol. Subsequently, medium was replaced every two days.

Immunocytochemistry

Coverslips were rinsed in phosphate buffered saline (PBS) and fixed with 4% paraformaldehyde for 30 min. After rinsing in PBS, the coverslips were incubated for one hour in blocking solution containing 1% 05-011 5 bovine serum albumin, 5% horse serum and 0.5% Triton in PBS. The following antibodies were used at the indicated dilutions: β-III-tubulin/Tuj-I (1:600, Promega), Neurofilament (1:600, Sigma), Peripherin (1:1100, Chemicon), Brn3a (1:100, Chemicon), TH (1:100, Chemicon), NCAM (1:200, Chemicon), p75 (undiluted, Santa Cruz), E-cadherin (1:60, NeoMarker), and AP2 (DSHB). Sections were rinsed repeatedly in PBS and then incubated with the primary antibodies for 1 h at room temperature or overnight at 4° C. Secondary antibodies (Alexa 488, 594, and biotinylated anti-mouse and anti-rabbit followed by Extravidin-conjugates) were then applied, and nuclei were stained with Hoechst (0.1 μg/ml). Coverslips were then rinsed in PBS, mounted on microscope slides in 90% glycerol, 10% PBS, 1% n-propyl-gallate, and sealed with nail polish.

RT-PCR Analysis

RNA was extracted using Tri-Reagent (Sigma #T9424) and the Aurum Total RNA kit (#732-6820 BIO-RAD), according to the manufacturers' protocols. Reverse transcription polymerase chain reaction (RT-PCR) was performed using Ready-To-Go RT-PCR beads (#27-9266-01 Amersham Biosciences) or Ready-Mix reverse-iT one step kit (#AB-0844/LD ABgene).

Photography

Preparations were viewed with an Olympus BX60 microscope, and photographed using a frame-grabber (Scion) and analogue video camera (Cohu). A Bio-Rad MRC 1000 confocal microscope was used for some photographs. Images were enhanced using ImageJ (NIH) and Paint-Shop-Pro (Jasc) software. 05-011 6

Quantitative Analysis

Four-week co-cultures were immunostained for peripherin (peri+) and Brn3a (brn+). Colonies were classified as: containing double-stained cells, (peri+/brn+), containing single-stained cells, or negative for both. A double-stained colony was one in which cells that express both Brn3a in the nucleus and peripherin in the cytoplasm were clearly identified. A few colonies were so thick, that it was not possible to determine accurately whether Brn3a and peripherin were in the same cells, so these were not included in the quantification. Three separate experiments were performed for quantitation purposes, and over 300 total colonies counted.

Results

SDIA-Induction of Neuron-Like Cells from HESC

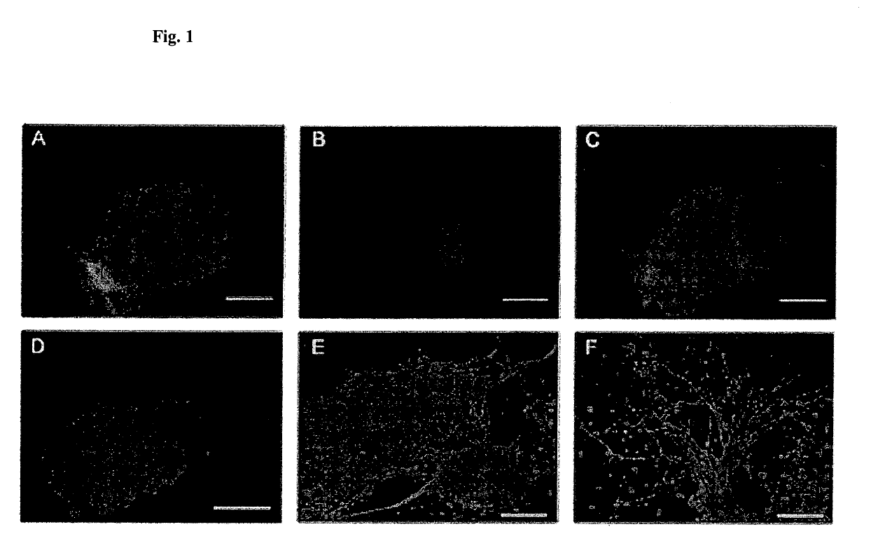

Cells with the molecular characteristics of neurons are induced from murine and primate ES when co-cultured with the mouse stromal line PA6 (Kawasaki et al., 2000, Neuron 28:31-40 and Mizuseki et al., 2003, PNAS 100:5828-5833). This experiment has been repeated three times with modified conditions and cultured two lines of HESC with PA6 cells (SDIA). After 7 days of culturing HESC with PA6, a distinct morphological change in the hESC colonies was observed. Undifferentiated HESC cultured with mouse or human foreskin fibroblasts appeared as dense, round, or oval monolayer colonies. By contrast, HESC colonies were irregular in outline after 7 days of SDIA-treatment. At this time point, the large majority of colonies differentiated into primarily NCAM+ neural precursor cells (FIG. 1A, B, C). Many colonies, primarily the larger ones, contained cells expressing the non-neural ectodermal marker E-cadherin+ in their centers (FIG. 1B, C). Several colonies expressed a few β-3-tubulin (Tuj-1)-staining cells (a universal and early marker for neurons), but most of these Tuj-1+ cells did not extend axons (not shown). The results are consistent with other observations that PA6 induces neural differentiation from HESC (Buytaert-Hoefen et al., 2004, Cell 22:669-674). Three weeks after seeding HESC on PA6 cells, massive neuronal differentiation was observed using immunohistochemisty. More than 50% of the colonies were Tuj-1+ with extensive networks of stained axons (FIG. 1E, F). When HESC were grown with the same media but on gelatin or laminin without a PA6 cell feeder layer, very few cells survived after two weeks of co-culture, and none expressed neural markers (not shown).

Generation of Peripheral Neuron-Like Cells from HESC

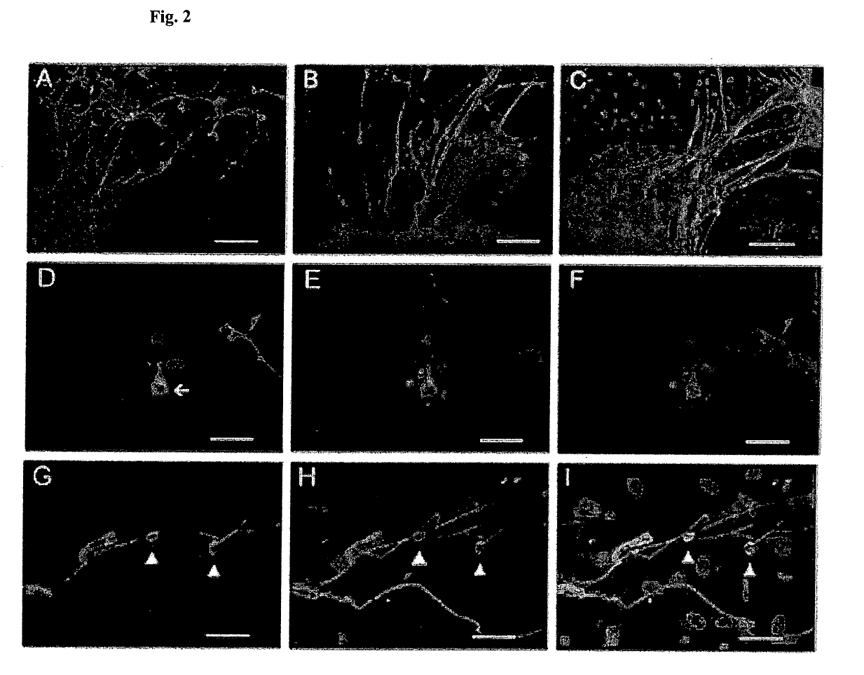

In order to ascertain whether peripheral neurons are present in SDIA-induced cultures, the cells were immunostained for the protein peripherin, which is present in neurons with axons outside the CNS (including sensory ganglion neurons, sympathetic ganglion neurons, and primary motoneurons; Troy et al., 1990, Neuroscience 36:217-237). After 4 weeks of SDIA treatment, 51% of the colonies contained Tuj-1+/peri-CNS-like neurons and 34.5% contained Tuj-1+/peripherin+ HPN-like neurons (402 colonies, 3 independent experiments) (FIG. 2A, B). The number of peripherin+/Tuj-1+ cells was very variable between the colonies. RT-PCR analysis confirmed that peripherin mRNA was expressed in 3 weeks, but not 1-week cultures (see below).

Most of the Tuj-1+ colonies also contained cells and processes expressing TH (FIG. 2C). Several neuronal types express TH, including catecholaminergic CNS neurons and peripheral sympathetic ganglion neurons. In order to explore whether the TH+ neurons in the SDIA cultures were CNS or HPN-like, we double-stained colonies induced for 3 weeks with SDIA for peripherin and TH, a combination characteristic of sympathetic neurons. Both peri+ and peri-(FIG 2D-I) populations were present in the cultures, but most of the TH+ cells-were TH+/peri CNS-like catecholaminergic neurons.

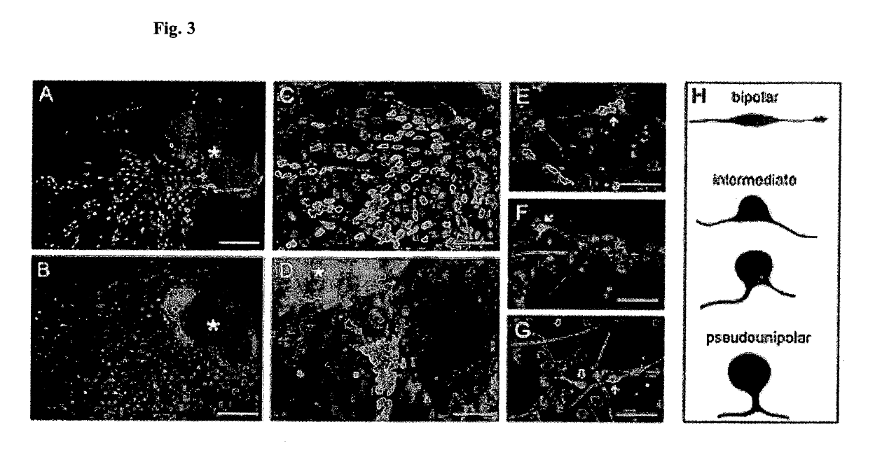

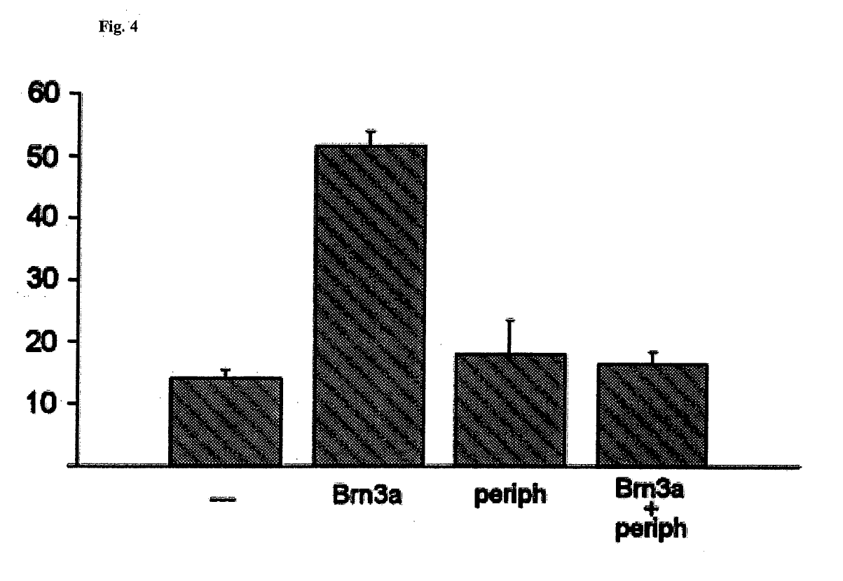

The cultures were next tested for the presence of another peripheral neuron sub-population, sensory ganglion neurons (PSGN). There is no single marker thought to be specific for PSGN. Therefore, in previous studies of PSGN differentiation, double-staining with antibodies to Brn3a (a transcription factor characteristic of PSGN and a small population of CNS neurons; Fedstova et al., 1995, Mech. Dev. 53:291-304) and peripherin has been used as a criterion for PSGN identity (Mizuseki et al., 2003, PNAS 100:5828-5833). This combination of antibodies was used to stain the cultures induced by SDIA for four weeks. Cells positive for both Brn3a and peripherin were observed in some of the colonies (FIG. 3). Some of these putative sensory neurons migrated away from the colonies, as might be expected from neurons derived from the migratory neural crest. The percentage of colonies that contained cells expressing Brn3a, peripherin, and double-stained cells was determined in three separate experiments. Quantitative analysis revealed that double-stained cells were present in 16.5% of the more than colonies observed, approximately the same proportion of colonies that contained peripherin+-only cells. About half of the colonies contained Brn3a+ cells, and a small percentage did not contain staining for either marker (FIG. 4). Double-stained neurons were also observed at three weeks, but not at two weeks of co-culture (not shown).

Immature PSGN (like many other neurons) are bipolar, and the majority of the peri+/brn3a+ neurons observed had this morphology (FIG. 3E,G). However, mature PSGN have a unique pseudounipolar structure, this morphology arising from the fusion of the proximal segments of the two initial processes (FIG. 3H). A few pseudounipolar peri+/brn+ cells were observed as well as a number of double-stained cells with morphologies intermediate between immature and mature sensory neurons (FIG. 3E-G). Peri+/brn+ cells with more than two processes exiting from the soma were never observed, in contrast to the frequent Tuj-1+ and TH+/peri+ multipolar neurons.

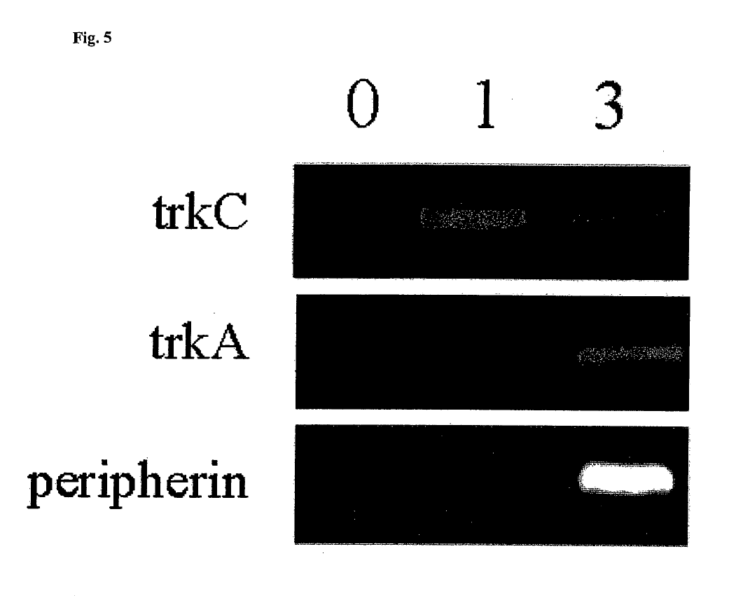

PSGN express tyrosine-kinase receptors (Trks) that bind trophic factors of the neurotrophin family (reviewed in Huang and Reichardt, 2001, Annu. Rev. Neurosci. 24:677-736). In the chick embryo, TrkC is initially expressed in migrating neural crest and in virtually all cells in the nascent dorsal root ganglia (DRG), and then is down-regulated in most DRG cells, and only remains in large, proprioceptive neurons (Kahane and Kalcheim, 1994, J. Neurobiol. 25:571-584). By contrast, TrkA is only expressed in apparently post-mitotic neurons in DRG, and appears somewhat later than TrkC (Rifkin et al., 2000, Dev. Biol. 227:465-480). RT-PCR analysis of SDIA-treated HESC revealed that TrkC was induced in one-week co-cultures when compared to naïve hESC, but, subsequently, its expression was lower at three weeks of co-cultures (FIG. 5). TrkA, by contrast, was expressed only at low levels in the 1-week co-cultures, and was highly induced in the 3-week cultures. Although other cell types express these receptors, these results are consistent with the known pattern of expression of these receptors in developing PSGN in the chick embryo. Peripherin mRNA was first observed at 3 weeks of co-culture, as was the case for the protein (FIG. 5).

SDIA-Induces Differentiation of NC-Like Cells

Most HPN neurons develop from the neural crest in vertebrate embryos (with the exception of those derived from ectodermal placodes in the head). In order to determine whether the differentiation of the HPN-like neurons we observed might be preceded by NC-like cells, we examined 7-day SDIA-induced HESC for the presence of molecules expressed in murine NC. Unfortunately, there is no one specific marker indicative of neural crest identity, so we examined a number of different molecules used as HNCC markers in several species by immunostaining and RT-PCR.

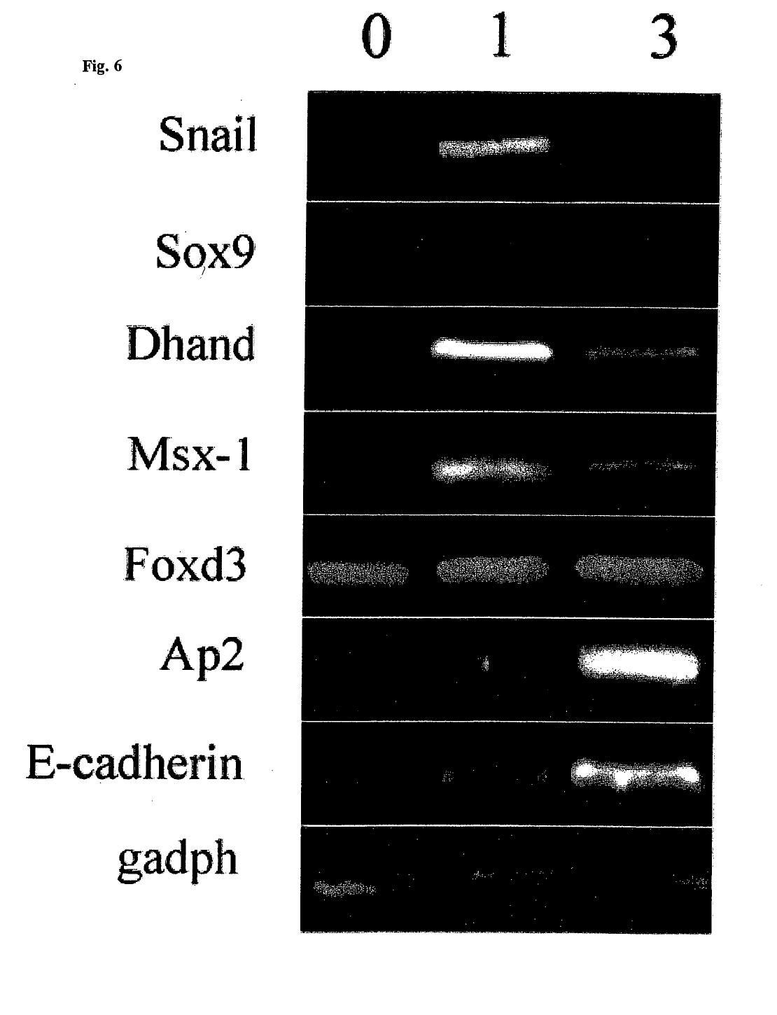

RT-PCR analysis was performed for a series of HNCC markers on undifferentiated HESC, and on HESC after 1 and 3 weeks SDIA treatment. High expression of the early mammalian (Locascio et al., 2002, PNAS 99:16841-16846) HNCC cell marker (Nieto et al., 1992, Development 116:227-237) SNAIL was observed after one week of SDIA-induction, and was dramatically reduced at 3 week of treatment (FIG. 6). Other transcripts associated with HNCC development in the mouse also induced by one week of SDIA-treatment included Sox9, dHAND, and MSX1. The up-regulation of these genes in the 1-week cultures, and the subsequent fall in their expression by 3 weeks of culture, are consistent with the presence of neural crest-like cells in the 1-week cultures, and their subsequent differentiation into sensory-like and sympathetic-like neurons by the third week.

A few genes considered to be neural crest markers did not show this pattern of early up-and later down-regulation. FoxD3, expressed in the pre-migratory neural crest, was present in the 1-week co-cultures. However, it was also expressed in naïve HESC (FIG. 6 and Mitchell et al., 1991, Genes Dev. 5:105-119) as well as at three weeks of culture, so its detection was less informative than the aforementioned transcripts as to the presence of NC-like cells in the cultures. Immunocytochemistry showed that AP2 was expressed after 1 week of SDIA treatment, and this was confirmed by the presence of its mRNA. However, AP2 is expressed by epidermal cells as well as NC, and it therefore not surprising that its mRNA expression continued to rise until 3 weeks, similarly to the increase in E-cadherin expression. It is therefore likely that our culture conditions are permissive for epidermal cells to differentiate and/or multiply. Pigmented cells were not observed in the cultures, and no staining was done for smooth muscle actin, because the SDIA method, coupled with the differentiation medium used, has already been shown to inhibit the production of melanocytes and mesenchymal HNCC derivatives (Mizuseki et al., 2003, PNAS 100:5828-5833).

Immunocytochemical evidence also supported the induction of HNCC from HESC using SDIA. Cells expressing AP2+/NCAM were present after one week of co-culture, a combination thought to be characteristic of HNCC cells (FIG. 1D and Mitchell et al., 1991, Genes Dev. 5:105-119). Most of the colonies also immunostained positive for cells that expressed the low affinity neurotrophin receptor p75, which is expressed in migrating murine crest cells (Stemple and Anderson, 1992, Cell 71:973-985) (not shown).

PCR primers were designed to be specific for human mRNAs. Control experiments showed that the (murine) PA6 cells grown alone did not express any of the mRNAs for human HNCC markers. The PA6 cells expressed murine, and not human actin transcripts, as expected (not shown).

SDIA-Induces Differentiation of Schwann Cells

SCIA-induced HESC cultures were grown as described supra. RT-PCR revealed that protein zero mRNA, a schwann cell specific transcript, was present at one week of SDIA treatment and increased at three weeks SDIA treatment.

### Example 2

### Generation of Peripheral Sensory Neurons from Neurospheres

Neurospheres were cultivated for 3 weeks in feeder-free conditions with presence of noggin (700 ng/ml).

About 20 neurospheres were gently trypsinized to a single cell suspension and plated on cover slips with PA6 cells in DRG medium (Glutamine 1%, Penicillin streptomycin 1%, B27 supplement 2%, DMEM/F12 97%, NGF 10 ηg/ml). Medium was replaced every 3 days. After 7 days of co-culture with PA6 cells, a morphological change was observed; dissociated neurospheres treated with SDIA produced heterogeneous colonies, and frequently processes were seen outgrowing from the colonies (data not shown). After 26 days of co-culturing, neural differentiation was seen using immunohistochemisty. 39.5% of colonies were immunopositive for peripherin, 14.5% of colonies were immunopositive for brn3a, and 23.7% of colonies expressed brn3a+ peripherin markers(indication for the presence of sensory neurons).

### Example 3

### Purification of Neural Crest Cells

Human neural crest cells as prepared in Example 1 are isolated using a monoclonal antibody to low affinity NGF receptor (p75) essentially as performed for murine neural crest cells in Stemple et al., 1992, Cell 71:973-85. Briefly, a population of cells comprising neural crest cells is incubated with a monoclonal antibody which specifically binds to the neural crest cell-specific low affinity NGF receptor (p75). Cells to which the monoclonal antibody is bound are purified by any method known in the art. For example, the cells are further incubated with a fluorescence conjugated antibody that binds to the low affinity NGF receptor antibody and cells are subjected to FACS. Alternatively, the cells are further incubated with an antibody that binds to the low affinity NGF receptor antibody that is attached to a solid matrix (e.g., magnetic bead or matrix of a columns). The purity of the population is assayed by determining the per cent of purified cells that express a neural crest cell specific marker.

### Example 4

### Generation of HESC Lines Stably Transfected with a Selection Marker-Reporter Gene Under the Control of a Cell-Type-Specific Promoter

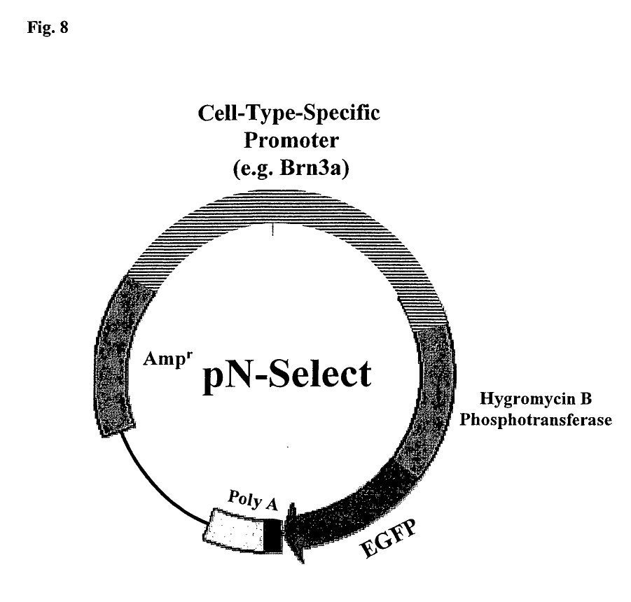

The reporter-selection marker construct pN-Select (FIG. 8) is derived by replacing the constitutive cytomegalovirus promoter from pHygEGFP (Clontech), with a cell-type specific promoter, in this case, the Brn3a promoter. This promoter will be silent in transfected HESC and thus cells that do not possess Brn3a promoter activity will be killed by contact with the selection agent hygromycin in the concentration range of 100 to 200 μg/ml. The vector pPUR (Clontech) is an expression vector in which puromycin N-acetyl-transferase expression is driven by the constitutive SV40 promoter. Both plasmids are linearized and HESC are co-transfected, by electroporation, with pN-Select vector and pPUR vector at a molar ratio of approximately 15:1 (pN-Select:pPUR).

HESC are removed in intact clumps using collagenase IV (1 mg/ml; Invitrogen) for 7 minutes, washed with cell culture medium, and resuspended in 0.5 ml of cell culture medium (1.5-3.0×10⁷ cells). Prior to electroporation, approximately 40 μg of mixed linearized pN-Select and pPUR plasmid DNA, in 0.3 ml of PBS, is added to the HESC suspension. Cells are electroporated with one pulse in an electroporator (BioRad) set to 320V, 200 μF, in a 0.4 cm gap cuvette, at room temperature. After electroporation, cells are incubated for 10 minutes at room temperature and are then plated at high density in a 10 cm cell culture dish coated with Matrigel. Puromycin selection (3 μg/ml) is started 48 hours after electroporation. Puromycin-containing medium was replaced every 3-4 days. After three weeks surviving colonies are picked, expanded, and tested in neural differentiation experiments. Peripheral neurons expressing Brn3a are resistant to hygromycin and exhibit EGFP fluorescence.

1. A method for inducing differentiation of human embryonic stem cells (HESC) or human neural progenitor cells (HNPr) into populations of differentiated neuronal cells comprising co-culturing said HESC or HNPr under non-aggregation conditions with stromal cells or stromal cell derived components under serum-free conditions, wherein at least one neuronal cell marker is present after six days in co-culture.

2. The method of claim 1 wherein said HESC are selected from the group consisting of HES1, HES2, HUES 1, HUES2, HUES3, HUES4, and HUES 7.

3. The method of claim 1 wherein said HNPr is selected from the group consisting of neurospheres and human neural crest cells (HNCC).

4. The method of claim 1 wherein said differentiated neuronal cells are selected from the group consisting of human peripheral neurons (HPN) and human schwann cells (HSC).

5. The method of claim 1 wherein said differentiated neuronal cells are HNC differentiated from HESC.

6. The method of claim 1 wherein said HESC or HNPr are further contacted with bone morphogenic protein 4, wnt, or retinoic acid.

7. The method of claim 1, wherein said HESC or HNPr overexpress NCX, Snail, FoxD3, Sox 9, beta-catenin, or neuregulin (GGF).

8. The method according to claim 1 wherein said stromal cells are selected from one of the following:
a) PA6 stromal cell line; or
b) a stromal cell line effective for inducing neural differentiation of human neural progenitor cells.

9. The method according to claim 6 wherein said stromal cells are mitotically inactivated by a method selected from the following:
a) contacting said stromal cells with mitomycin C at a concentration effective for blocking cell proliferation;
b) irradiating said stromal cells with a dose of γ-radiation effective for inhibiting cell proliferation; and
c) paraformaldehyde fixation.

10. The method according to claim 1 wherein said stromal cell derived component is selected from the group consisting of the following:
a) a stromal cell membrane preparation;
b) a stromal cell membrane preparation treated with a histological fixative; and
c) conditioned medium or purified component thereof from growth of stromal cells.

11. A method of purifying a desired differentiated neuronal cell comprising:
a) differentiating said neuronal cell from HESC or HNPr according to claim 1 wherein said that HESC or HNPr comprise a reporter gene operably associated with a cell type specific prompter, wherein said promoter is expressed in the desired differentiated neuronal cell; and
b) isolating cells that express said reporter gene.

12. The method of claim 11 wherein said desired differentiated neuronal cell is a HNCC and said promoter is selected for the group consisting of Snail, Sox 9, Msx 1, dHAND, and low affinity NGF receptor (p75).

13. A method for inducing differentiation of a neural crest cell-derived cell from a neural crest cell comprising:
a) isolating said HNCC of claim 1;
b) incubating said HNCC with at least one factor that causes HNCC differentiation or overexpressing in said HNCC at least one factor that causes HNCC differentiation.

14. The method of claim 13, wherein said neural crest cell-derived cell is selected from the group consisting of neurons, glia, secretory cells of the peripheral neuroendocrine system, melanocytes, chondrocytes, and smooth myocytes.

15. A method of treating a disorder associated with deficient or defective differentiated neuronal cells comprising administering to a patient in need thereof differentiated neuronal cells that have been differentiated from human embryonic stem cells (HESC) or human neural progenitor cells (HNPr) by the method comprising co-culturing said HESC or HNPr under non-aggregation conditions with stromal cells or stromal cell derived components under serum-free conditions, wherein at least one neuronal cell marker is present after six days in co-culture.

16. The method of claim 15 wherein said disorder is familial dysautonomia and said deficient or defective differentiated neuronal cells are peripheral neurons.

17. A method of screening for an agent that alters differentiation of neuronal cells comprising:
a) co-culturing said HESC or HNPr with under non-aggregation conditions with stromal cells or stromal cell derived components under serum-free conditions in the presence of the candidate compound
b) determining the affect on differentiation of the HESC or HNPr to the differentiated neuronal cell,
wherein an alteration of the differentiation of the HESC or HNPr to the differentiated neuronal cells as compared to the differentiation of HESC or HNPr not contacted with the agent to the differentiated neuronal cells indicates that the candidate compound alters differentiation.

18. A method for generating an in vitro model of human familial dysautonomia comprising
a) generating HESC or HNPr comprising a modification such that expression levels of properly spliced IκBKAP polypeptide are decreased relative to wild type cells;
b) co-culturing said HESC or HNPr with under non-aggregation conditions with stromal cells or stromal cell derived components under serum-free conditions; and
c) isolating peripheral neuronal cells wherein said peripheral neuronal cells comprise lower expression levels of properly spliced IκBKAP polypeptide.

19. The method of claim 18 wherein said modification is selected from the group consisting of:
a) altering the endogenous IκBKAP gene on one or more of the HESC or HNPr chromosomes to make a mutant IκBKAP gene;
b) introducing a mini-gene comprising a mutated IκBKAP gene into the HESC or HNPr; and
c) using siRNA to decrease expression of the IκBKAP gene.

20. The method of claim 19 wherein said mutant IκBKAP gene comprises a IVS20^{+6T→C} transversion.
